(19) United States
(12) Patent Application Publication
(10) Pub. No.: US 2013/0309178 A1
(43) Pub. Date: Nov. 21, 2013

(54) COMPOSITIONS FOR AEROSOLIZATIONS OF HIGHLY CONDUCTIVE SOLUTIONS

(71) Applicants: Michael D. TRIPLETT, New Albany, OH (US); Bruce D. McVeety, Dublin, OH (US); Christopher Rulison, Burton, OH (US)

(72) Inventors: Michael D. TRIPLETT, New Albany, OH (US); Bruce D. McVeety, Dublin, OH (US); Christopher Rulison, Burton, OH (US)

(21) Appl. No.: 13/896,628

(22) Filed: May 17, 2013

Related U.S. Application Data
(63) Continuation of application No. 12/525,485, filed on Feb. 22, 2010, now abandoned.
(60) Provisional application No. 60/899,235, filed on Feb. 1, 2007.
(60) Provisional application No. 60/927,243, filed on May 1, 2007.

(51) Int. Cl. A61K 9/12

(52) U.S. Cl. CPC A61K 9/12
USPC 424/43

(57) ABSTRACT

Compositions for aerosolization of conductive solutions (preferably highly conductive solutions) by way of electrostatic or electrohydrodynamic spraying are provided. Methods for making and using these compositions and electrostatic and electrohydrodynamic aerosol generators containing these compositions are also provided.

### BACKGROUND OF THE INVENTION

A. Field of the Invention

The present invention relates to compositions for aerosolization of highly conductive liquid compositions (formulations) by way of electrostatic or electrohydrodynamic spraying, as well as methods for making and using these compositions. In some embodiments, the conductive compositions may be used in combination with electrostatic spraying devices capable of delivering a small particle size (e.g., 1-5 μm). The highly conductive liquid compositions of the invention comprise three or more basic components: an active agent; liquid carrier material(s) in which the active ingredient may be dissolved, suspended, or emulsified; and aerosol adjusting material(s) which provide the compositions with surface rheological properties which enable the production of an aerosol generation cloud by electrostatic or electrohydrodynamic (EHD) means.

B. Description of Related Art

Devices and methods for forming fine sprays by particular electrostatic techniques are known. For example, U.S. Pat. No. 4,962,885 to Coffee, incorporated by reference herein, describes a process and apparatus to form a fine spray of electrostatically charged droplets. More specifically, the process and apparatus comprise a conductive nozzle charged to a potential on the order of 1-20,000 volts, closely adjacent to a grounded electrode. A corresponding electric field produced between the nozzle and the grounded electrode is sufficiently intense to atomize liquid delivered to the nozzle, and thereby produce a supply of fine charged liquid droplets. However, the field is not so intense as to cause corona discharge, resulting in high current consumption. Uses of such liquid dispenser processes and apparatuses include sprayers for paint and spraying of crops. Aerosolization of liquids using electric fields is often referred to as electrostatic aerosolization.

More recently, there has been recognition that such spraying devices are useful for producing and delivering aerosols of therapeutic products for inhalation by patients. In one particular example, described in U.S. Pat. No. 6,302,331 to Dvorsky et al. incorporated by reference herein, fluid is delivered to a nozzle that is maintained at high electric potential relative to a proximate electrode to cause aerosolization of the fluid with the fluid emerging from the nozzle in a conical shape called a Taylor cone. One type of nozzle used in such devices is a capillary tube that is capable of conducting electricity. An electric potential is placed on the capillary tube which charges the fluid contents such that the fluid emerges from the tip or end of the capillary tube in the form of a Taylor cone. The Taylor cone shape of the fluid before it is dispensed results from a balance of the forces of electric charge on the fluid and the fluid's own surface tension. Due to the finite conductivity of most liquids, a thin liquid (on the order of 1 μm diameter) jet (with speeds up to 10 m/s) emerges from the cone tip. Due to Rayleigh instability, the jet breaks up into a stream of mono-dispersed charged particles. The charge then causes the droplet stream to diverge into a conical aerosol spray. [LJ.09] The Dynamics of a Steady Taylor cone electrospray Martin Bell (Ohio University, Athens, Ohio 45701), Maarten A. Rutgers (The Ohio State University, Columbus, Ohio 43210) ; Session LJ—Surface Tension Effects I. ORAL session, Tuesday morning, November 24, Jefferson, Adam's Mark Hotel . The resulting aerosol spray may remain charged or can be discharged to produce a neutral spay. Studies have shown that this aerosol (often described as a soft cloud) has a uniform droplet size and a high velocity leaving the tip but that it quickly decelerates to a very low velocity a short distance beyond the tip.

Electrostatic sprayers produce charged droplets at the tip of the nozzle. Depending on the use, these charged droplets can be partially or fully neutralized (with a reference or discharge electrode in the sprayer device). The typical applications for an electrostatic sprayer, without means for discharging or means for partially discharging an aerosol would include a paint sprayer or insecticide sprayer. These types of sprayers may be preferred since the aerosol would have a residual electric charge as it leaves the sprayer so that the droplets would be attracted to and tightly adhere to the surface being coated. However, in other cases it may be preferred that the aerosol be completely electrically neutralized. For example, in the delivery of some therapeutic aerosols electric neutralization or discharge allows the aerosol to impact deep in the lung rather than adhere to the linings of the mouth and throat.

At the present time, inhalation therapy is a rapidly evolving technology. Numerous active drugs are being developed with the expectation that effective delivery of and treatment with these drugs will be possible by means of inhaled aerosols. Aerosolizing active ingredients requires a composition with certain characteristics and properties that make the composition compatible with the aerosolization process. The process of formulating particular active ingredients, such as drugs, with the appropriate liquid carriers, such as organic solvents, can be particularly challenging. Therefore, there is a need for basic or general compositions which are compatible with a variety of active ingredients, a range of suitable carriers, and appropriate aerosol generating devices.

U.S. Pat. No. 4,829,996 to Noakes et al., U.S. Pat. No. 5,707,352 to Sekins et al. and U.S. Pat. No. 6,503,481 to Browning et al., each of which is incorporated by reference herein, all disclose formulations suitable for use with electrostatic aerosol devices; however, despite this prior art, spraying highly conductive formulations (solutions where the conductivity is around or greater than 12.5 μS/cm) remains challenging in the cone-jet mode at relatively high volumetric flow rates and low voltage requirements. Typically, spraying highly conductive formulations yields large particles, multi-modal distributions, and numerous discharge streamers. This undesirable outcome results from an imbalance between the electrical and physiochemical forces within the Taylor cone.

While it is possible to aerosolize highly conductive formulations without altering surface rheology by reducing the fluid's volumetric flow rate well below what would be practical for many applications including pulmonary therapeutics it would be highly desirable to use an EHD deice to aerosolize highly conductive formulations at high flow rates and at relatively high conductivities.

Hartman et al. [J. Aerosol Sci. Vol. 30, No. 7, pp. 823-849, 1999] discusses a force balance on an idealized Taylor cone under laminar flow conditions. Within the Taylor cone, the electrical forces of normal electric stress, tangential electric stress, and electric polarization stress resulting from the electric charge are balanced against the physiochemical forces of surface tension, viscosity, and gravity. As liquid conductivity increases (i.e. resistivity decreases), the electrical forces increase while the physiochemical forces, with surface tension being the most important, remain constant. Thus, the resulting force imbalance induces Taylor cone instability, leading to poor spraying. The force imbalance can lead to complex chaotic flow behavior [Marginean, I., Nemes, P. and Vertes, A., Order-Chaos—Order Transitions in Electrosprays: The Electrified Dripping Faucet, Physical Review Letters 11 Aug. 2006, PRL 97, 064502 (2006)].

Although aerosolization of highly conductive liquid formulations may prove challenging when using electrostatic or EHD aerosolization, highly conductive formulations may be required for the delivery of certain active ingredients to the desired target surface. For example, small molecular salts are typically more soluble in conductive solvents such as water or alcohols. In addition, other active ingredients are inherently highly conductive (i.e., ionic species, including some peptides and proteins). Some active ingredients (and formulations) require pH stabilization and or solubilization of the active agent which makes the solution conductive. Other additional materials such as aerosol adjusting materials may be ionic or charged species. Thus, there are many instances when the ability to electrostatically aerosolize highly conductive liquid formulations enables the delivery of active ingredients that could otherwise not be delivered electrostatically.

Accordingly, to compensate for the force imbalance resulting from highly conductive liquids, it is desirable to make the fluid surface more viscous as represented by a low surface overall viscoelastic modulus and phase angle in a manner that does not significantly increase the fluid viscosity.

The inventors have discovered that by controlling critical surface viscoelastic properties, one is able to increase the efficiency (as measured by the combination of flow rate and applied voltage) of spraying conductive formulations using an EHD aerosolization means.

### SUMMARY OF THE INVENTION

The present invention is directed to liquid compositions that include highly conductive carrier liquids, where the composition is capable of being aerosolized into small uniform particles. According to some embodiments, a predetermined quantity of a desired ingredient can be delivered to a site of choice (e.g., an active pharmaceutical ingredient to the lungs of the user) with an electrostatic or electrohydrodynamic aerosol generating device. Critical properties of the composition are provided for desirable, and preferably optimal, use of the composition with such aerosol generating devices. The compositions according to some embodiments of the present invention may contain two, three or more basic components that may be present in a variety of combinations, concentrations, and ratios to one another.

In some embodiments of the present invention, the first component of the composition is an active ingredient. The active ingredient or agent may be any agent or mixture of agents that is to be delivered as an aerosol to a target surface. Examples of such agents are agricultural chemicals, paints, cosmetics and pharmaceuticals. In the agricultural field, the active agent may be an insecticide, fungicide, a herbicide or mixtures of such agents. In the pharmaceutical field, the active agent may be a therapeutic drug, biologically active protein or peptide, or a vaccine.

The second component of the therapeutic composition is a liquid carrier material in which the active ingredient may be dissolved, suspended, or emulsified; examples of such carrier liquids include water, alcohols, ethers, alkyl sulfoxides and combinations thereof. In particularly preferred embodiments the solvent is alcohol, preferably ethanol in combination with glycerol. In some cases, it may be desirable to use mixtures of liquid carrier materials to achieve the degree of conductivity that is needed to achieve optimal aerosolization of the liquid.

Additional components of the liquid composition (which may be optional in some embodiments) include material(s) responsible for adjusting the surface rheological properties of the liquid composition within ranges specified for optimal aerosolization (e.g., ranges that produce small uniform droplets with an electrostatic or electrohydrodynamic device).

It may be appreciated that the present invention provides compositions of highly conductive liquid compositions which have certain preferred characteristics. These preferred characteristics cause aerosols generated from the compositions to also have particular preferred characteristics. A typical embodiment of this invention includes a liquid composition having predetermined surface rheological properties which facilitate aerosolization of the composition with an EHD aerosolization device.

Further objects, advantages, and novel aspects of this invention will become apparent upon consideration of the subsequent detailed description.

### BRIEF DESCRIPTION OF THE DRAWING

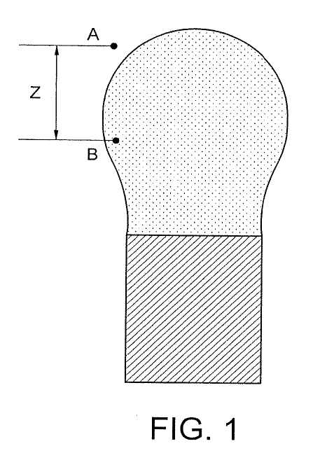

FIG. I is a simplified representation of a pendant bubble; in FIG. 1, A and B are discrete points on the surface of the bubble and Z is the vertical distance between the two points.

### DETAILED DESCRIPTION OF THE INVENTION

The present invention is directed to a highly conductive liquid composition capable of aerosolized using an electrostatic or electrohydrodynamic aerosol generating device wherein the highly conductive liquid composition has a surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm and preferably from about 12.5 μSiemens/cm to about 400 μSiemens/cm.

Yet another embodiment of the present invention is directed to an aerosol formed when a highly conductive liquid composition is aerosolized using an electrostatic or electrohydrodynamic aerosol generating device wherein the highly conductive liquid composition has surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm and preferably from about 12.5 μSiemens/cm to about 400 μSiemens/cm.

The invention is further directed to a highly conductive liquid composition capable of aerosolization using an electrostatic or electrohydrodynamic aerosol generating device wherein the highly conductive liquid composition has surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm and preferably from about 12.5 μSiemens/cm to about 400 μSiemens/cm·0.5 mN/m to about 10 mN/m, wherein said liquid composition is comprised of:
i) one or more active agents;
ii) a carrier liquid;
iii) one or more materials for adjusting the surface rheological properties of said liquid composition; and
iv) optionally, one or more formulation additives.

A further embodiment of the invention is directed to a highly conductive liquid composition for direct delivery of an active drug to a target surface in need of treatment comprising an effective amount of said active agent dissolved, suspended or emulsified in a liquid carrier vehicle, wherein the highly conductive liquid composition has surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm and preferably from about 12.5 μSiemens/cm to about 400 μSiemens/cm; wherein said liquid carrier may optionally contain the following components:
i) one or more materials for adjusting the surface rheological properties of said liquid carrier; and
ii) one or more formulation excipients.

The highly conductive liquid compositions according to the present invention are compatible with an electrostatic or electrohydrodynamic aerosol-generating device so that an aerosol cloud with certain preferred properties and characteristics is produced each time the device is used. As used herein, both electrostatic and electrohydrodynamic devices are collectively referred to as “EHD” devices.

Aerosols having uniformly-sized particles and uniform distribution patterns are desirable over aerosols that do not possess these characteristics because they exhibit more desirable deposition properties such as for inhalation into the pulmonary tract of the user (i.e., where they have a higher respirable fraction). When used with compatible compositions, electrostatic and EHD aerosol generating devices can be adjusted to create substantially mono-modal aerosols having particles more uniform in size than aerosols generated by other devices or methods.

Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. In other devices, one electrode may be highly charged, whereas the second electrode is grounded. For example, the discharge electrode could be at + or −10 kV while the nozzle is grounded. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric tangential and normal forces exerted on the surface of the droplet overcome the surface tension of the liquid, thereby generating a stream of liquid that disperses into many smaller droplets of roughly the same size. These smaller droplets form a mist which constitutes the aerosol cloud that the user ultimately inhales.

As used herein, the term “active agent” refers to the material which is delivered to a target surface by means of an aerosol formed when the highly conductive liquid compositions of the invention are sprayed, i.e., aerosolized by means of an EHD device. Electrostatic or EHD aerosolization can be used to distribute an active agent to a desired site (target Surface). For example, in spray painting, it is preferred to deliver the optimal amount of pigment and binder to the surface to be coated and not to any surrounding areas. In other embodiments, EHD aerosolization of highly conductive liquid compositions solutions may be used to deliver agricultural chemical such as insecticides, fungicides, cosmetics, e.g., liquid foundation and tanning formulations, flavoring agents and pharmaceutically active agents.

The highly conductive liquid composition of some embodiments of the present invention may contain at least one active ingredient at a concentration permitting delivery of the desired amount of active agent to the target surface. As would be recognized by one skilled in this art, the number and types of active agents suitable for delivery to a target surface by means of an aerosol varies widely and includes numerous options.

When the active agent is a pharmaceutical, the composition may include at least one active ingredient selected from the following: insulin and other proteins, salt forms of active ingredients, small molecule and synthetic drugs; vaccines; nucleic acids, including DNA and RNA vectors and vaccines; aptamers; gene therapy agents for treating diseases such as cystic fibrosis; enzymes, hormones; antibodies; vitamins; peptides and polypeptides; oligonucleotides; cells; antigens; allergens; anti-infectious agents including antimicrobials, antibiotics, antifungals and antivirals; anti-cancer agents; and pain management drugs such as narcotics.

Particularly suitable drugs for use herein include albuterol (also known as salbutamol), atropine, budesonide, cromolyn, epinephrine, ephedrine, fentanyl, flunisolide, formoterol, ipratropium bromide, isoproterenol, pirbuterol, prednisolone, triamcinolone acetonide, salmeterol, amiloride, fluticasone as well as the pharmaceutically acceptable acid addition salts and esters of the foregoing drugs, their hydrates and their other solvates.

Other suitable medicaments for use in the compositions and methods of the invention include, antineoplastic agents, such cisplatin and carboplatin, methotrexate, taxol, mitomycin, bleomycin, vincristine, vinblastine, dactinomycin, daunorubicin, doxorubicin, mithramycin, tamoxifen, etoposide, alpha- and beta-interferon; anti-fungal agents such as ketoconazole, nystatin, and amphotericin B; beta-lactam antibiotics; hormones such as human growth hormone; steroids, e.g., hydrocortisone and prednisone; vitamins e.g., retinoic acid and derivatives such as 13-cis-retinoic acid; peptides, such as insulin, interferons and interleukins; antivirals such as acyclovir, and azidothymidine (AZT); antibiotics such as chloramphenicol and clindamycin; anti-inflammatories; opiates; sedatives; and local anesthetics such as lidocaine hydrochloride.

As used herein, the term “pharmaceutically active agent” refers to biologically active agents that are administered to human or animal patients as the active drug substance for treatment of a disease or condition. Such active drug substances are administered to a patient in a “pharmaceutically effective amount” to treat a disease or condition. A suitable medicament or drug is one which is suitable for administration by inhalation, the inhalation being used for oral and nasal inhalation therapy.

As would be recognized by one skilled in the art, by “pharmaceutically effective amount” is meant an amount of a pharmaceutically active agent having a therapeutically relevant effect on the disease or condition to be treated. A therapeutically relevant effect relieves to some extent one or more symptoms of the disease or condition in a patient or returns to normal either partially or completely one or more physiological or biochemical parameters associated with or causative of the disease or condition. Specific details of the dosage of a particular active drug may be found in its labeling, i.e., the package insert (see 21 CFR §201.56 & 201.57) approved by the United States Food and Drug Administration.

When the term “effective amount” is used in connection with an active agent that is not a pharmaceutical, the effective amount of a 1 particular active agent will depend on the nature of the active agent and the reason one is delivering the active agent to a target surface.

When an active agent is added to the liquid carrier a solution is produced if the active agent is soluble in the liquid carrier and a suspension is produced if the active agent is insoluble. The term “suspension” as used herein is given its ordinary meaning and refers to particles of active agent or aggregates of particles of active agent suspended in the liquid carrier. When the active agent is present as a suspension the particles of active agent will preferably be in the nanometer range; e.g. from about 10 nm to about 2500 nm; preferably from about 50 nm to about 1000 nm and more preferably from about 50 nm to about 500 nm. In the case where the active agent is a pharmaceutical, in order to assure formation of good aerosols and aerosol deposition in the lungs, it is important that the particle size of the drug be less than the size of the aerosol droplets. If the carrier liquid is an emulsion containing a continuous and discontinuous phase, the active agent may be dissolved or suspended in one of the phases.

The liquid carrier vehicles of the invention are useful for preparing aerosols for the delivery of pharmaceutically active agents to the “respiratory tract” of a patient using an EHD spraying/aerosolization device. The term “respiratory tract” as used herein includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli, which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems, 6: 273-313, (1990).

Usually, the deep lung, or alveoli, is the primary target of inhaled therapeutic aerosols for systemic delivery. However, as used herein, the term “respiratory tract” is additionally meant to include administration of the medicament compositions of the invention to the mucosa of the nasal passages and to the mucosa of the bucca. The preferred target for systemic delivery of an aerosol of an active agent is the deep lung or alveoli.

The particle size of the aerosol droplets produced when the liquid carrier described herein is sprayed with an EHD device will range from about 1 μm to about 300 μm in diameter and preferably from about 1 μm to about 50 μm in diameter. As would be recognized by one skilled in the art, the particle size of the aerosol will be selected depending on the use of the aerosol; as an example the particle size of aerosolized paint will in general be larger than that of an aerosolized drug delivered to the deep lung. On the other hand, the particle size of a cosmetic such as a tanning composition should be greater than about 50 μm in diameter to prevent respiration of the aerosol particles by the individual being sprayed.

If the aerosol is being administered to the respiratory tract of a patient if the drug is to be delivered to the deep lung for systemic activity, the particle size of the resulting aerosol will range from about 1 μm to about 8.0 μm and preferably from about 1 μm to about 5.0 μm. If the drug is to be delivered to the mid-lung, the particle size of the resulting aerosol will range from about 2 μm to about 10 μm and preferably from about 5 μm to about 10 μm will be used. If the pharmaceutically active agent is delivered to the oropharangeal region the particle size of the aerosol will generally range from about 2 μm to about 10 μm with a range of from about 5 μm to about 10 μm being preferred. If the drug is to be delivered to the buccal mucosa or to the nares, the particle size of the resulting aerosol will range from about 10 μm to about 50 μm and preferably from about 20 μm to about 30 μm will be used.

Delivery of a drug to the pulmonary tract of an animal by aerosolization may be preferable to other methods of drug delivery in certain circumstances. Delivery of drugs or other active ingredients directly to the patient's lungs provides numerous advantages including: providing an extensive surface area for drug absorption, direct delivery of therapeutic agents to the disease site in the case of regional drug therapy, eliminating the possibility of drug degradation in the patient's intestinal tract (a risk associated with oral administration), and eliminating the need for repeated subcutaneous injections. Furthermore, delivery of drugs to the pulmonary system by means of aerosol inhalation may be used to deliver drugs systemically, as well as for targeted local drug delivery for treatment of respiratory ailments such as lung cancer or asthma. Moreover, electrostatic-type inhalers, in which the charge on the droplets is typically neutralized, have demonstrated advantages over more conventional metered dose inhalers (MDI) including producing more uniform droplets, enabling the patient to inhale the formed aerosol liquid or mist with normal aspiration, producing higher dosage efficiencies, and providing more reproducible doses. Important considerations in administering an aerosolized active ingredient to the lungs of a patient include the characteristics of the composition containing the active ingredient and the aerosol cloud that will ultimately be inhaled by the patient or user. Compositions according to some embodiments should be able to be consistently sprayed through an aerosol-generating device, and should be well-tolerated by the user The compositions include a suitable carrier for the active ingredient. In addition, the active ingredient according to some embodiments should be stable for a period of time in the composition.

Furthermore, the aerosol-generating device itself should effectively and consistently convert the formula into an aerosol cloud with certain desired properties. For example, an aerosol-generating device should not deliver a high velocity aerosol which makes it difficult for the user to inhale aerosol particles. Preferred aerosol characteristics also include an aerosol cloud composed of particles that are roughly uniform in size. An aerosol cloud composed of uniform particles of a predetermined size typically provides the most efficient and effective delivery of the therapeutic composition to the patient or user because the dosage that the patient receives can be more precisely controlled (i.e., uniform particle size equals more precise delivery and dosage). Therefore, for maximum effectiveness of both drug and aerosol device, consistent generation of uniformly sized aerosol particles typically should occur each time the composition is aerosolized with a particular device.

As used herein, the term “target surface” refers to the surface upon which the aerosol particles produced when an EHD device is used to aerosolize the highly liquid compositions of the invention will be deposited. Illustrative examples of such target surfaces are surfaces being painted, foliage of plants, the face and/or body of a person to liquid makeup is being applied, the body of a person to whom tanning material is being applied, to a wound on the body of a human or animal, and in the case of a drug being delivered to the pulmonary tract of a patient the oropharynx, larynx, trachea, bronchi, bronchioli and the alveoli.

As used herein, the term “carrier liquid” refers to a highly conductive liquid in which the active ingredient may be dissolved, suspended or emulsified and which is highly conductive. Since the electrical forces play such an important role in determining the aerosol formation in EHD spraying it is understandable to one skilled in the art that highly conductive liquids may behave significantly differently than relatively non-conductive liquids. In order for liquid formulations to be sprayed electrostatically, the formulations should be somewhat conductive. As used herein, the term “highly conductive” liquid formulations of the invention refer to liquid compositions of high conductivity having conductivities greater than about 12.5 μS/cm. The conductivities may range as high as (or even higher than) 400 μS/cm.

A variety of solvents or mixtures of solvents may be suitable for use as a carrier liquid. For example, in some embodiments of the present invention, either water or ethanol (depending on the solubility characteristics of the active ingredient) is used as the solvent in which the active ingredient is dissolved or suspended. In general, the carrier liquid (solvent) may be selected from the group consisting of alcohols, ethers, alkyl sulfoxides, perfluorocarbons and hydrofluoroalkanes and combinations of such solvents. Since water and ethanol are relatively benign to the atmosphere and to humans and animals if inhaled, mixtures of these solvents are frequently used as the carrier liquid.

In some embodiments, the carrier (solvent) fraction of the composition may represent 5 to 95% (v/v) of the total volume of the liquid composition. In other embodiments, the fraction of the liquid composition represented by the carrier liquid varies depending on the solubility or insolubility of the active ingredient. For example, if an active ingredient is highly soluble in the carrier (e.g. water), then the carrier fraction of the total composition may be as low as about 5.0% to 10.0% (v/v). If an active ingredient is only moderately soluble in water (or other carrier liquid), a larger fraction of the carrier liquid may be required to completely dissolve or sufficiently suspend the active agent. In other embodiments, use of a mixture of liquids, e.g., ethanol and glycerol may be required to obtain a stable composition that can be sprayed. For example, the carrier may be any mixture of water and ethanol, or water and propylene glycol or ethanol and water and glycerol, etc., or various combinations of these liquid carriers.

In a preferred embodiment of the present invention, the solvent(s) selected as carrier liquids are chosen based both on compatibility with certain active ingredients and on their compatibility with EHD devices, and typically include water and/or ethanol.

The present inventors have discovered that when critical surface viscoelastic modulus and phase angle values are satisfied, EHD aerosol generators are capable of generating aerosols in which particle size, aerosol velocity, and the resultant deposition patterns can be more precisely controlled. EHD aerosol generators, in which the resulting aerosol particles are not charged, are ideal devices for use with therapeutic compositions that are to be delivered to a patient's pulmonary system by inhalation. When such critical values are satisfied, electrostatic aerosol generating devices can also produce desirable spray properties for highly conductive liquid formulations.

In some embodiments of the present invention, relevant surface rheological characteristics of the composition include surface viscoelastic modulus (E₁ₛ) in units such as milliNewtons per millimeter (mN/m) and phase angle (δ₁ₛ) in units such as degrees. Surface viscoelastic modulus is a measure of the extent that a liquid's surface tension deviates from its original state relative to a perturbation. A low surface viscoelastic modulus represents a fluid surface that resists surface perturbations in an analogous manner to shock absorbers on a car. More precisely, the surface tension changes minimally in response to a change in surface area. In the case of highly conductive formulations, the surface perturbations are thought to result from localized electrical force fluctuations and the rapid creation of new surface as the fluid elongates to form a Taylor cone.

The surface viscoelastic modulus (E) of the highly conductive liquid compositions of the invention will range from about 0.5 mN/m to about 10 mN/m; preferably from about 2.0 mN/m to about 7.5 mN/m; and more preferably about 5.0 mN/m.

Phase angle is a measure of the time required for the surface to respond to a perturbation. A large phase angle indicates a slower surface response to a perturbation. A slower surface response is considered desirable. In the inventions described herein, the phase angle will range from about 0.5 degrees to about 90 degrees and preferably from about 10 degrees to about 50 degrees and more preferably about 25 degrees.

For electrostatic spraying of highly conductive compositions it is preferred that the solution has both a low overall surface viscoelastic modulus (E) and high phase angle (δ) at short oscillation periods, particularly at a 1 s oscillation period, to facilitate aerosolization of highly conductive formulations. In practical terms, the surface becomes more viscous as E decreases and δ increases. As E decreases, the surface tension increases less during drop expansion. As δ increases, surface tension increases are dampened more effectively. Essentially, the more viscous surface acts as a shock absorber to the increased, variable electrical stresses (due to increased electrical charge in the fluid) on the Taylor cone surface.

In a preferred embodiment, E should be less than 10 mN/m and more preferably be less than 7.5 mN/m while phase angle (δ) should preferably be more than 10 degrees and more preferably be more than 20 degrees.

Surface tension is a property possessed by liquid surfaces whereby these surfaces behave as if covered by a thin elastic membrane in a state of tension. Surface tension is a measure of the energy needed to increase the surface area of the liquid. Liquids with a lower surface tension will aerosolize more easily than liquids with higher surface tension.

Surface tension is measured by the force acting normally across unit length in the surface. The phenomenon of surface tension is due to unbalanced molecular cohesive forces near the surface of a liquid. As this term is used herein, it refers to the surface tension of the liquid formulation in the Taylor Cone just before formation of aerosol droplets.

In some embodiments of the present invention, the surface tension of the liquid composition is within the range of from about 10 to about 72 milliNewtons/meter. In more preferred embodiments of the present invention, the surface tension of the composition is within the range of from about 15 to about 45 milliNewtons/meter. In most preferred embodiments of the present invention, the surface tension of the composition is within the range of from about 20 to from about 35 milliNewtons/meter.

Viscosity is the measure of the resistance to fluid flow; thus liquids that flow easily generally have lower viscosity. The viscosity of a liquid composition is not affected significantly by the addition of small amounts of active agent to the composition. However, the addition of certain suspending agents or very high concentration of an active agent can increase the viscosity of the liquid composition. Viscosity may not be a key parameter in forming the aerosols of the present invention, but it does affect particle size distribution. Highly viscous materials tend to form aerosols larger particle sizes and with more disperse or bimodal distributions. Beyond a critical viscosity value, the formed jet emanating from the Taylor cone will not break up into discreet aerosol particles and instead will form continuous ligaments.

In some embodiments of this invention, the surface rheological properties of the liquid composition comprise: a surface viscoelastic modulus that is preferably less than 10 mN/m, and a Phase angle that is greater than 10 degrees while the conductivity is between 12.5 and 1000 μSiemens/cm and preferably from about between 12.5 μSiemens/cm to about 400 μSiemens/cm. In some embodiments, it may be possible to achieve a liquid composition with physical properties falling within these parameters by simply combining the active ingredient and the carrier material(s). However, if the combination of the active ingredient and the carrier material does not produce a liquid composition having physical properties falling within these parameters, the addition of surfactant(s) and/or polymer(s) to the solution will bring the composition within the required parameters.

Highly conductive liquids can be stabilized by the use of materials that adjust surface rheological properties. Agents such as film forming agents, surfactants, polymers, proteins, peptides, and biopolymers may be used to adjust the surface rheological properties. Film forming agents such as polyvinylpyrrolidone (PVP) polymers, cellulosic materials such as hydroxypropyl methylcellulose, and other film forming agents, and mixtures of the same may be used herein.

The polymer(s) that may be added to the composition include PVP polymers of various molecular weights such as PVP 40K, tyloxapol, polyethylene glycol, triton, and biopolymers such as hydroxypropyl methylcellulose and hydroxy methylcellulose, and combinations thereof.

It has been discovered that certain surface rheological properties of the liquid compositions of the invention are critical in obtaining stable, mono-modal aerosols of the highly conductive formulations with an EHD device. Therefore, according to some embodiments of the present invention, a surfactant or multiple surfactants may be added to the active ingredient and carrier liquid to adjust the carrier liquid's surface rheological characteristics.

Surfactants such as natural and synthetic phospholipid derivatives, e.g., lecithin, 1-palmitoyl-2-(16-fluoropallmitoyl)-sn-glycero-3-phosphocholine (DPPC) and 1,2-dimyristoylamido-1,2-deoxyPhophotidylcholine (DDPC); polysorbates, e.g., polyoxyethylene sorbitan monooleate (Tween 80), sorbitan monooleate (Span 80), sorbitan trioleate (Span 85); oleic acid; polyols such as glycerol; medium chain triglycerides; fatty acids; soybean oil; olive oil; sodium dodecyl sulfate (SDS); modified sugar surfactants; and combinations of the such surfactants have been found to be useful in the highly conductive liquid formulations of the invention.

Combinations of these surfactant material(s) and/or polymer(s) described herein are advantageous in some embodiments of the invention. For example, the use of ethanol alone may create an aerosol, but the particle size of the aerosol may be below the preferred range. By combining ethanol and polyethylene glycol in a predetermined ratio to one another, the preferred particle size can be achieved.

The addition of surfactants and/or polymers to the carrier liquid can alter surface rheological parameter and bring the liquid composition back within the desired and preferably optimal ranges. Addition of surfactants and/or polymers is necessary only in embodiments of the present invention in which the combined active ingredient(s) and solvent(s) material do not yield an aerosol with the desired characteristics. In some embodiments of the invention, surfactants and/or polymers are present in the liquid compositions at from about 0.05% to about 50% weight percent (w/v) of the liquid composition.

Depending on the application the aerosols of the invention are being used, additional formulation excipients may be included in the composition. Such materials may be included for a variety of purposes including but not limited to: stabilization of the liquid composition; facilitating control of aerosol particle size; increasing the solubility of the active ingredient in the composition; lowering the surface tension of the liquid; antimicrobial, antioxidant and the like. As would be recognized by those skilled in the art, additional ingredients may be added as long as the resulting liquid formulation has the following critical properties: i.e., a surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm and preferably from about 12.5 μSiemens/cm to about 400 μSiemens/cm.

Once solubilized, suspended or emulsified, the active ingredient should also be stable in the liquid carrier itself, and stable in the final composition. Stability requires that the active ingredient not lose activity prior to aerosolization (i.e., retains a reasonable shelf-life), and that the active ingredient not lose activity or degrade significantly as a result of the process of aerosolization. Furthermore, in some applications it is required that the highly conductive liquid composition be stable over time. In various embodiments, stability issues can be addressed by the addition of a stabilizing ingredient to the composition.

One or more of the following ingredients may be added to the formulations of the invention to increase physical stability of the composition: oils, glycerides, polysorbates, celluloses lecithin, polyvinyl pyrrolidone, polyethyl glycol, saccharide gums, and alginates. In some embodiments, antioxidants such as ascorbic acid and ascorbic acid esters, Vitamin E, tocopherols, butylated hydroxyanisole, and butylated hydroxytoluene may be added to reduce degradation of an active agent such as a drug caused by oxidation. In some embodiments of the present invention, chelating or complexing agents such as citric acid, cyclodextrins, and ethylenediaminetetracetic acid may be added (as an alternative or in addition to the stabilizing agents just described) to stabilize drug compositions and to increase the solubility of the active ingredient in the composition.

Alternatively or additionally, in some embodiments preservative ingredients may be added to the composition to maintain the microbial integrity of the highly conductive composition. For example, in some embodiments of the present invention, at least one of the following ingredients is added to preserve compositions against microbial contamination or attack: benzalkonium chlorides, phenol, parabens, or any other acceptable antimicrobial or antifungal agent.

In the case where the active agent is a drug, excipients may be added to the composition to enhance or increase a patient's ability to receive the aerosolized composition. For example, in some embodiments of the present invention, sugars or sugar alcohols such as sucrose, trehalose, and mannitol may be added either to stabilize compositions containing proteins, or to serve as sweeteners to improve the taste of the composition. In some embodiments, flavoring agents such as sugars, oils, citric acid, menthol, and camphor may be added to improve the flavor of a composition.

The following liquid compositions were prepared having the composition listed in Table I. These examples are meant to be illustrative of embodiments of the present invention, and are not meant to limit the full breadth of the invention disclosed herein. Each of the liquid formulations of Table I had the surface viscoelastic modulus and phase angle measured, as described below in Example 1 and as summarized in Table III.

TABLE I
Examples of Formulations of the Invention

| | FORMULATION 1 | |
|---|---|---|
| H₂O | 4.5% | v/v |
| EtOH | 85.5% | v/v |
| Glycerol | 10.0% | v/v |
| | FORMULATION 2 | |
| EtOH | 90.0% | v/v |
| Glycerol | 10.0% | v/v |
| Oleic Acid | 0.25% | w/v |
| PVP (40K) | 0.25 | w/v |
| | FORMULATION 3 | |
| EtOH | 90.0% | v/v |
| Glycerol | 10.0% | v/v |
| Oleic Acid | 0.45% | w/v |
| Span 80 | 0.08 | w/v |
| | FORMULATION 4 | |
| EtOH | 100.0% | v/v |
| Oleic Acid | 0.46% | w/v |
| Tween 80 | 0.54 | w/v |
| | FORMULATION 5 | |
| EtOH | 95.5% | v/v |
| Glycerol | 0.05% | v/v |
| Oleic Acid | 0.33% | w/v |
| Tween 80 | 0.67 | w/v |
| | FORMULATION 6 | |
| EtOH | 90.0% | v/v |
| Glycerol | 10.0% | v/v |
| Oleic Acid | 0.33% | w/v |
| Tween 80 | 0.67 | w/v |

Tiotropium Bromide 0.8 mg/ml of solution

In addition to the formulations in Table I, certain drugs were formulated and were successfully aerosolized. Table II lists the drugs, and other information about the formulation.

TABLE II
Examples of Aerosolized Drugs at Therapeutic Doses

| | Molecule | Formulation | | |
|---|---|---|---|---|
| Compound | Type | Type | Chemical Class | Indication |
| Fluticasone | Small | Solution | Corticosteroid | Asthma |
| Fluticasone - | Small | Solution | Corticosteroid - LABA | Asthma/COPD |
| Salmeterol | | | | |
| Fentanyl | Small | Solution | Opiate | Pain |
| Δ9-THC | Small | Solution | Cannabinoid | Pain/Wasting/Antiemesis |
| Zolmitriptan | Small | Solution | 5HT1B/1D Agonist | Pain |
| Granisetron | Small | Solution | 5HT3 Antagonist | Antiemesis |
| Partner NCEs | Small | Solution | Undisclosed | Respiratory Therapy |
| Rimonabant | Small | Solution | Cannabinoid Antagonist | Smoking |
| | | | | Cessation/Obesity |
| Estradiol | Small | Solution | Estrogen | Hormone Replacement |
| Scopolamine | Small | Solution | Anticholinergic | Antiemesis |
| Teriparitide | 4.1 kDa | Solution & | Peptide | Osteoporosis |
| | | Suspension | | |
| Budesonide | Small | Solution | Corticosteroid | Asthma |
| Iloprost | Small | Solution | Prostacyclin Analogue | Pulmonary Hypertension |
| Lidocaine | Small | Solution | Na+ Channel Blocker | Pain/Cough |
| Insulin | 5.8 kDa | Solution & | Peptide | Diabetes |
| | | Suspension | | |
| Tiotropium Br | Small | Solution | Muscarinic Antagonist | COPD |
| Tacrolimus | Small | Solution | Macrolide Antibiotic | Immunosuppression |

The formulations of Table I were each sprayed with an EHD hand-held aerosolization device described in U.S. patent applications U.S. Provisional Patent Application No. 60/773,272 An Accurate Metering System (U.S. utility filing application Ser. No. 10/560,540, filed Nov. 16, 2006) and U.S. Provisional No. 60/773,239 Dissociated Discharge EHD Sprayer with Electric Field Shield (U.S. utility filing application Ser. No. 11/560,542, filed Nov. 16, 2006) and U.S. application Ser. No. 11/485,787 Improved Dispensing Device and method (each of the foregoing applications herein incorporated by reference). The voltage and current required for optimal aerosolization was determined and summarized in Table III.

The aerosols produced when the liquids of Table I were sprayed using the voltage indicated in Table III were evaluated subjectively. The aerosol was visually observed for the qualities of wetness, the presence of “streamers”, plume width, pulsation, and micro-amperes (μA). A subjective aerosol score of 1, 3, or 5 was assigned to each aerosol, with a score of 1 being equal to poor performance, 3 equaling average performance and 5 equaling excellent performance. The score for each of the aerosols from the formulations described in Table I are shown in Table III.

Additional details regarding the surface rheological parameters described throughout this disclosure are provided in the following experimental description.

### EXAMPLE I

### Viscoelastic Modulus Evaluation

Surface rheology evaluation focuses on the perturbation of a surface which is at equilibrium initially. In this case, oscillation experiments were performed of the surface area of bubbles pre-formed within each of the liquid samples of Table I. This was done on a Tracker Oscillating Drop Tensiometer from IT Concept France. For each experiment, a bubble of specific surface area (in this case 25 mm²) is formed pendant on an upward pointing capillary within a bulk of the liquid. The bubble's surface tension and surface area are monitored optically by the pendant drop technique (see below) as the drop surface area is controllably oscillated (in this case by 12.5 mm² (50%) at various rates of oscillation.

Pendant drop surface tension experimentation operates as follows. A bubble of air is formed on an upward-pointing capillary tip within the liquid to be studied for surface tension. The bubble surface is then digitally imaged using a high pixel CCD camera. The bubble's image is then mathematically analyzed to determine its mean curvature at over 300 points along its surface as well as its surface area. The surface area data is used as feedback to a pump connected to the capillary which serves to add or subtract air from the bubble to control the bubble's surface area as desired.

The curvature data are used to determine the current surface tension. The curvature of a bubble which is pendant to a capillary tip, at any given point on its surface, is dependent on two opposing factors (or forces): buoyancy works to make the bubble elongated or “drip-like” in the upward direction; and surface tension works to keep the bubble spherical—since a sphere has the lowest surface to volume ratio of any shape. Surface tension by definition is the amount of work necessary to create a unit area of surface.

Accordingly, pendant drop surface tension evaluation involves observing the balance that exists between these two forces on a pendant bubble in the form of the bubble's mean curvature at various points along its surface with the continuous phase. Lower surface tension means a more “drip-like” bubble shape; higher surface tension means a more spherical drop shape.

The mathematics of pendant drop analysis are based on the Laplace equation which states that the pressure difference at any given point on the surface (ΔP) is equal to the mean curvature of the surface at that point ((1/r₁+1/r₂), where r₁ and r₂ are the principal radii of curvature) multiplied by twice the tension (σ) contained in the surface.

ΔP=(1/r₁+1/r₂)2σ

For a pendant bubble, the pressure difference within the drop, between any two vertical positions is:

Δρ g Z

where Δρ=the difference in density between the air that is forming the bubble and bulk liquid, g=gravity, and Z=the vertical distance between the two positions, as shown in FIG. 1.

Since the measurement of surface tension is actually made by determining the mean curvature on the drop at over 300 points (like those labeled A and B in FIG. 1), and the points are then used in pairs, with the equations given above, to solve for surface tension. In the following manner:

((1/r₁+1/r₂)_{at A}−(1/r₁+1/r₂)_{at B})2σ=Δρ g Z_{between A and B}

surface tension is determined at least 150 times on any given drop image. These surface tension values are averaged to give a single value for the overall surface tension of the drop. This technique has been found to be extremely accurate for determining surface tensions of liquids with known surface tension (typical errors of less than 0.1%).

The data from the Viscoelastic modulus and phase angle testing described is summarized in Columns 8 and 9 of Table III.

As the drop is controllably oscillated in terms of surface area, the surface tension response is monitored. Five (5) raw data files from this work have been provided, one for each sample studied. In each, the perturbation (or area strain) sine wave is the same, that is 50% of the initial surface area of 25 mm², first up to 37.5 mm² and then down to 12.5 mm². So the surface was both stretched and compressed. This was done at five oscillation periods: 1, 2, 5, 10, and 20 seconds for the complete oscillation.

In each experiment, the resultant surface tension wave was analyzed relative to the surface area perturbation wave using a simple Kelvin rheology model with an elastic (spring) element and a viscous (dashpot) element in series in order to obtain values of the overall viscoelastic modulus (E), the elastic modulus (E′) and the viscous modulus (E″) of the surface.

This is done as follows:

Viscoelastic Modulus (E)=dσ/(dA/A)

Elastic Modulus (E′)=E cos(δ)

Viscous Modulus (E″)=E sin(δ)

wherein:
dσ=amplitude of the surface tension sine wave relative to the equilibrium tension
dA/A=amplitude of the area oscillation relative to the initial area, in this case (37.5-25.0)/25.0=0.5 for all experiments
δ=the phase angle by which the surface tension response lags behind the area change perturbation. When δ=0° the response in perfectly elastic. When δ=90° the response is completely viscous. In any other condition, the response is viscoelastic.

TABLE III

| | | | | | | | Surface | Phase | |
|---|---|---|---|---|---|---|---|---|---|
| | | | | | | | Viscoelastic | Angle | |
| Example | Conductivity | Nozzle | Voltageₒₚₜ | Currentₒₚₜ | | | Modulus | δ₁ₛ | Subjective |
| No. | microS/cm) | Material | (kV) | micro(A) | Dv(50) | Span | E₁ₛ (mN/m) | (degrees) | Aerosol Score |
| 1 | 100.0 | PC | 11.0 | 50 | | | 12.0 | 4.1 | 1 |
| 2 | 102.7 | PC | 12.0 | 80 | | | 9.4 | 15.7 | 5 |
| 3 | 102.5 | PTFE | 11.5 | 65 | | | 9.5 | 16.3 | 5 |
| 4 | 95.7 | PTFE | 11.0 | 50 | 6.3 | 2.5 | 7.6 | 22.3 | 5 |
| 5 | 100.4 | PTFE | 11.2 | 45 | 4.9 | 3.1 | 6.3 | 25.6 | 5 |
| 6 | 38.0 | PTFE | 11.5 | 45 | 3.9 | 1.4 | 5.3 | 27.3 | 5 |

Nozzle Spray Qualification Checklist:
1 = poor performance
3 = average performance
5 = excellent performance

All of the articles, patents, patent applications, and/or publications recited in the present application are incorporated by reference in the present application in their entireties.

Thus it is seen that compositions and methods are provided for making and using conductive liquid compositions. Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made without departing from the spirit and scope of the invention as defined by the claims. Other aspects, advantages, and modifications are considered to be within the scope of the following claims.

1. A highly conductive liquid composition capable of aerosolization using an EHD aerosol generating device, wherein said highly conductive liquid composition has a surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm.

2. The highly conductive liquid composition according to claim 1 wherein the surface viscoelastic modulus of said liquid composition ranges from about 2.0 mN/m to from about 7.5 mN/m.

3. The highly conductive liquid composition according to any of the preceding claims 1-2, wherein the surface viscoelastic modulus of said liquid composition is about 5.0 mN/m.

4. The highly conductive liquid composition according to any of the preceding claims 1-3, wherein the phase angle of said liquid composition ranges from about 10 degrees to about 50 degrees.

5-16. (canceled)

17. An aerosol formed when a highly conductive liquid composition is aerosolized using an EHD aerosol generating device wherein the highly conductive liquid composition has a surface viscoelastic modulus of from about 0.5 mN/m to about 10 mN/m, a phase angle of from about 0.5 degrees to about 90 degrees, and a conductivity of from about 5.0 μSiemens/cm to about 1000 μSiemens/cm; wherein said highly conductive liquid composition comprises a liquid carrier vehicle; an active agent, wherein an effective amount of said active agent is dissolved, suspended or emulsified in said liquid carrier vehicle; and wherein said liquid carrier vehicle may optionally contain (i) one or more materials for adjusting the surface rheological properties of said liquid carrier vehicle, and (ii) one or more formulation excipients.

18. The aerosol formed when a highly conductive liquid composition is aerosolized according to claim 17 wherein said aerosol has an aerosol particle diameter of from about 1.0 μm to about 300.0 μm.

19. The aerosol formed when a highly conductive liquid composition is aerosolized according to any of the preceding claims 17-18, wherein said aerosol has an aerosol particle diameter of from about 1.0 μm to about 50.0 μm.

20. The aerosol formed when a highly conductive liquid composition is aerosolized according to any of the preceding claims 17-19, wherein the surface viscoelastic modulus of said liquid composition ranges from about 2.0 mN/m to from about 7.5 mN/m.

21. The aerosol formed when a highly conductive liquid composition is aerosolized according to any of the preceding claims 17-20, wherein the surface viscoelastic modulus of said liquid composition is about 5.0 mN/m.

22. The aerosol formed when a highly conductive liquid composition is aerosolized according to any of the preceding claims 17-21, wherein the phase angle of said liquid composition is from about 10 degrees to about 50 degrees.

23. The aerosol formed when a highly conductive liquid composition is aerosolized according to any of the preceding claims 17-22, wherein the phase angle of said liquid composition is about 25 degrees.

24. The aerosol formed when a highly conductive liquid composition is aerosolized according to any of the preceding claims 17-23, wherein the surface viscoelastic modulus is from about 2.0 mN/m to about 7.5 mN/m, wherein the phase angle is from about 10 degrees to about 50 degrees, and wherein the conductivity is from about 5.0 μSiemens/cm to about 400 μSiemens/cm.

25. The aerosol formed when a highly conductive liquid composition is aerosolized according to any of the preceding claims 17-24, wherein the surface viscoelastic modulus is about 5.0 mN/m, wherein the phase angle is about 25 degrees, and wherein the conductivity is about 12.5 μSiemens/cm.

26. The aerosol formed when a highly conductive liquid composition is aerosolized according to any of the preceding claims 17-25, wherein the conductivity of said liquid composition ranges form about 10.0 μSiemens/cm to about 400 μSiemens/cm.

27. The aerosol formed when a highly conductive liquid composition is aerosolized according to any of the preceding claims 17-26, wherein the conductivity of said liquid composition is about 12.5 μSiemens/cm.

28. The aerosol formed when a highly conductive liquid composition is aerosolized according to any of the preceding claims 17-27, wherein said liquid composition has a surface tension ranging from about 10 milliNewtons/meter to about the 72.0 milliNewtons/meter and wherein said aerosol formed from said liquid composition has a particle size diameter of from about 1.0 μm to about 300.0 μm.

29. The aerosol formed when a highly conductive liquid composition is aerosolized according to any of the preceding claims 17-28, wherein said material for adjusting surface rheological properties comprises film forming agents, surfactants and polymers or mixtures thereof.

30. The aerosol formed when a highly conductive liquid composition is aerosolized according to any of the preceding claims 17-29, wherein said material for adjusting surface rheological properties is selected from the group consisting essentially polyvinylpyrrolidone (PVP) polymers, tyloxapol, triton, polyethylene glycol, hydroxypropyl methylcellulose and hydroxy methylcellulose or mixtures thereof.

31. The aerosol formed when a highly conductive liquid composition is aerosolized according to any of the preceding claims 17-30, wherein said active agent is dissolved or suspended in said liquid carrier vehicle.

32. The aerosol formed when a highly conductive liquid composition is aerosolized according to any of the preceding claims 17-31, wherein said formation excipient comprises an agent that increases physical stability of said liquid composition, an antioxidant, a chelating or complexing agent, an antimicrobial agent, an antifungal agent or a flavoring agent.

33-50. (canceled)
